(19) United States
(12) Patent Application Publication
(10) Pub. No.: US 2014/0348706 A1
(43) Pub. Date: Nov. 27, 2014

(54) Microfluidic Device and Microfluidic System

(71) Applicant: Agency for Science, Technology and Research, Singapo (SG)

(72) Inventor: Abdur Rub Abdur Rahman, Singapore (SG)

(21) Appl. No.: 14/344,770

(22) PCT Filed: Oct. 24, 2012
(86) PCT No.: PCT/SG2012/000400

§ 371 Date: Mar. 13, 2014

(30) Foreign Application Priority Data
Oct. 24, 2011 (SG) SG201107813-6

(51) Int. Cl. G01N 33/483

(52) U.S. Cl. CPC G01N 33/4836
USPC 422/73; 422/502

(57) ABSTRACT

A microfluidic device and a microfluidic system are provided. The microfluidic device includes a support substrate; and at least one filter array arranged on the support substrate and configured so as to trap a single cell in contact with the support substrate within the at least one filter array when a sample including a plurality of cells flows into the microfluidic device from a direction substantially perpendicular to a plane of the support substrate and flows out in a direction substantially parallel to the plane of the support substrate.

### TECHNICAL FIELD

Various embodiments relate generally to a microfluidic device and a microfluidic system. Various embodiments relate generally to a microfluidic device for single cell filtering and a microfluidic device for single cell filtering and detection.

### BACKGROUND

Circulating tumor cells (CTCs) can be used in cancer diagnosis, depending on the disease progression in patients, ranging from as low as 1 cell per ml of blood to a few hundreds. CTCs are gaining clinical significance and are FDA approved to be used as biomarkers for various types of cancers. CTC detection from whole blood primarily involves isolating CTCs from blood followed by counting or reverse transcription polymerase chain reaction (RT PCR) for molecular recognition of cells. Regardless of which approach is taken post-isolation, isolation with high efficiency and fidelity is desirable in CTC detection.

Various approaches for CTC isolation, which can be categorized as antibody based and size based, have been used. In antibody based approach, anti-Epithelial cell adhesion molecule (EpCAM) antibody is coated on magnetic beads or on micro-fabricated structures which are then exposed to CTCs. This results in immunogenic interaction between the CTCs and the anti-EpCAM substrate, leading to selective enrichment of CTCs. This method, although benefitting from readily available anti-EpCAM formulations on beads, suffers from heterogeneity in expression of EpCAM in different types of cancer cells and also suffers from the loss of cells in sample handling and processing.

Alternatively, CTCs can be enriched by exploiting their relatively large size in comparison to other cells found in blood and their deformability (relative rigidity) in relation to other blood cells. It has been shown that CTCs can be enriched with high efficiency by size based filtration using track etched filters of microfabricated filters. The high efficiency isolation of CTCs from whole blood has been demonstrated for a variety of cell types, including in clinical trials.

Sized based filtration e.g. using track etched filters usually involve filtration of blood using the filter followed by optical imaging or RT PCR. However, optical microscopy based identification and manual counting of CTCs may suffer from high expense, requirement of highly trained operators and operator-to-operator variance.

### SUMMARY

According to one embodiment, a microfluidic device for single cell filtering is provided. The microfluidic device includes a support substrate; and at least one filter array arranged on the support substrate and configured so as to trap a single cell in contact with the support substrate within the at least one filter array when a sample including a plurality of cells flows into the microfluidic device from a direction substantially perpendicular to a plane of the support substrate and flows out in a direction substantially parallel to the plane of the support substrate.

According to another embodiment, a microfluidic device for single cell filtering and detection is provided. The microfluidic device includes a sensor array including at least one support substrate and a plurality of sensing electrodes arranged spaced apart on the at least one support substrate; and a plurality of filter arrays arranged on the sensor array, each of the plurality of filter arrays arranged corresponding to each of the plurality of sensing electrodes and configured to trap a single cell in contact with each of the plurality of sensing electrodes within each of the plurality of filter arrays when a sample including a plurality of cells flows into the microfluidic device in a direction substantially perpendicular to a plane of the at least one support substrate and flows out in a direction substantially parallel to the plane of the at least one support substrate.

According to yet another embodiment, a microfluidic system is provided. The microfluidic system includes a microfluidic device, at least one pump, and at least one valve, wherein the at least one pump and the at least one valve is configured to allow a sample including a plurality of cells to be pumped into the microfluidic device.

### BRIEF DESCRIPTION OF THE DRAWINGS

In the drawings, like reference characters generally refer to the same parts throughout the different views. The drawings are not necessarily to scale, emphasis instead generally being placed upon illustrating the principles of the invention. In the following description, various embodiments of the invention are described with reference to the following drawings, in which:

FIG. 1 shows a schematic diagram of a microfluidic device according to one embodiment.

FIG. 2 shows a schematic diagram of a microfluidic device according to one embodiment.

FIG. 3 shows a three-dimensional view of a microfluidic device according to one embodiment.

FIG. 4 shows a three-dimensional view of a microfluidic device according to one embodiment.

FIG. 5 shows a three-dimensional view of a microfluidic device according to one embodiment.

FIG. 6 shows a schematic diagram of a microfluidic device according to one embodiment.

FIG. 7 shows a schematic diagram of a microfluidic device according to one embodiment.

FIG. 8 shows a schematic diagram of a microfluidic device according to one embodiment.

FIG. 9 shows a schematic diagram of a microfluidic device according to one embodiment.

FIG. 10a shows a three-dimensional view of a microfluidic device for simulation according to one embodiment.

FIGS. 10b and 10c show simulated flow velocity profiles of a sample in a microfluidic device according to one embodiment.

FIGS. 11a and 11b show simulated flow velocity profiles of a sample in a microfluidic device according to one embodiment.

FIGS. 12a to 12f show an exemplary process of manufacturing a microfluidic device according to one embodiment.

FIG. 13 shows a schematic diagram of a microfluidic system according to one embodiment.

FIGS. 14a to 14d show scanning electron micrographs of a microfluidic device according to one embodiment.

### DETAILED DESCRIPTION

Embodiments of a microfluidic device and a microfluidic system will be described in detail below with reference to the accompanying figures. It will be appreciated that the embodiments described below can be modified in various aspects without changing the essence of the invention.

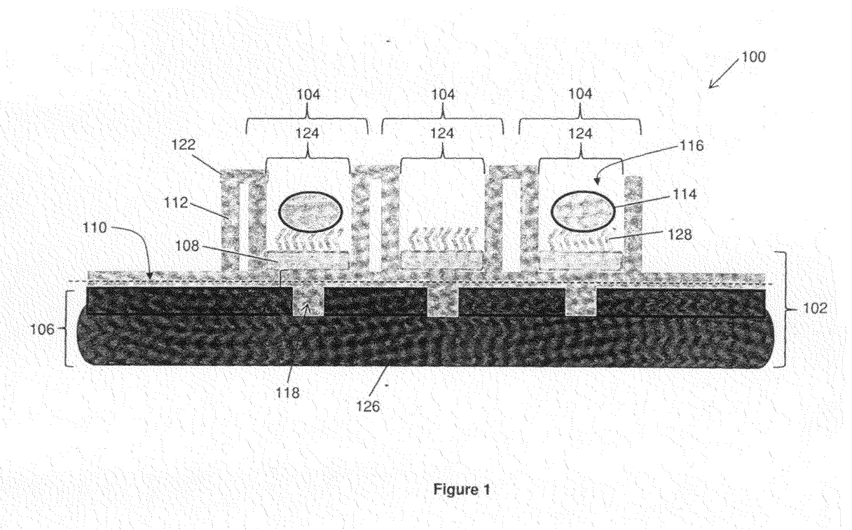

FIG. 1 shows a schematic diagram of a microfluidic device 100. The microfluidic device 100 may be used for single cell filtering and detection. The microfluidic device 100 includes a sensor array 102 and a plurality of filter arrays 104. The sensor array 102 includes at least one support substrate 106 and a plurality of sensing electrodes 108 arranged spaced apart on the at least one support substrate 106.

In one embodiment, the at least one support substrate 106 may have a plurality of support substrates arranged in a stack. The microfluidic device 100 may include an adhesive layer positioned between adjacent support substrates of the plurality of support substrates. The adjacent support substrates of the plurality of support substrates are bonded together by means of the adhesive layer. For illustration purposes, only one support substrate 106 is shown.

In one embodiment, the support substrate 106 includes silicon, glass, polymer (e.g. PMMA, PDMS, polycarbonate) and other materials that can be used for microfabrication, microelectronics and microfluidics. The sensing electrodes 108 include a conducting material. The conducting material may include but is not limited to gold, platinum and conducting polymer. The sensing electrodes 108 may include a semiconducting material. The semiconducting material may include silicon.

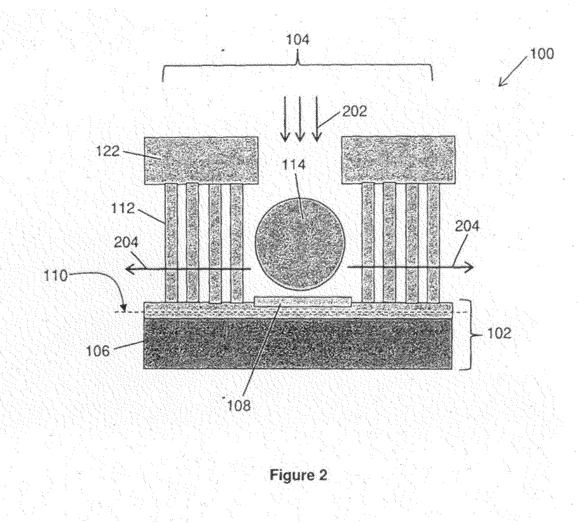

In one embodiment, the plurality of filter arrays 104 is arranged on the sensor array 102. The plurality of filter arrays 104 and the at least one support substrate 106 of the sensor array 102 may be integrated. Each of the plurality of filter arrays 104 is arranged corresponding to each of the plurality of sensing electrodes 108. Each sensing electrode 108 may be arranged within a corresponding filter array 104. Each of the plurality of filter arrays 104 is configured to trap a single cell 114 in contact with each of the plurality of sensing electrodes 108 within each of the plurality of filter arrays 104 when a sample (not shown) including a plurality of cells 114 flows into the microfluidic device 100 in a direction 202 substantially perpendicular to a plane 110 of the at least one support substrate 106 and flows out in a direction 204 substantially parallel to the plane of the at least one support substrate 106. The direction 202 and the direction 204 of the flow of the sample are illustrated in FIG. 2.

In one embodiment, each of the plurality of filter arrays 104 has a plurality of spaced apart filter pillars 112 extending from the plane 110 of the at least one substrate 106. The plurality of spaced apart filter pillars 112 is configured to substantially surround the single cell 114 in contact with each of the plurality of sensing electrodes 108.

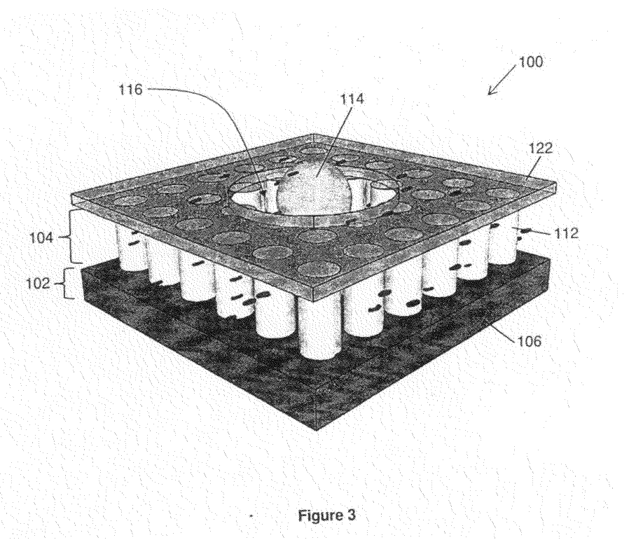

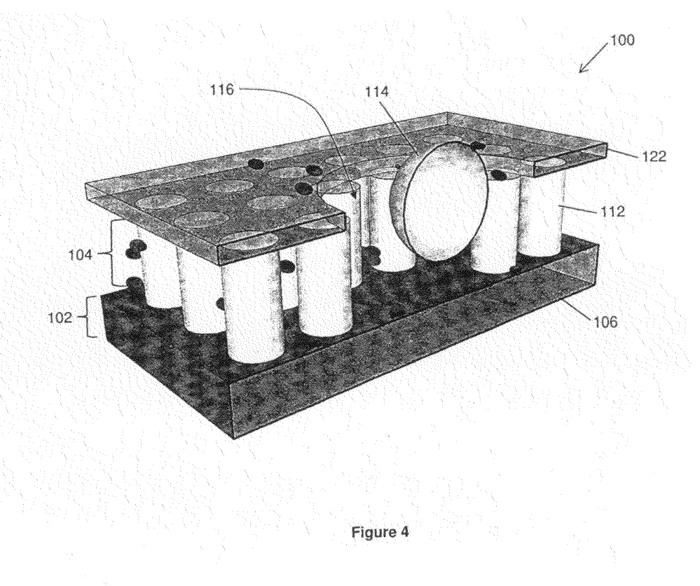

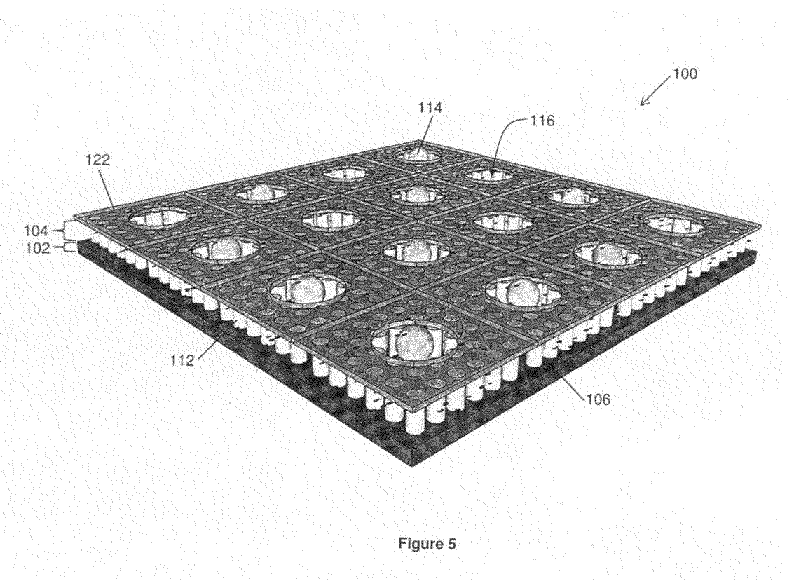

The arrangement of the filter pillars 112 of each filter array 104 are more clearly illustrated in three-dimensional views of the microfluidic device 100 in FIGS. 3 to 5. The filter pillars 112 of each filter array 104 are arranged to form a receptacle 116 for receiving the single cell 114 respectively.

The spacing between the filter pillars 112 of each filter array 104 can allow passage of deformable erythrocytes, leukocytes and other smaller cells while retaining the CTCs in the receptable 116 due to their large size. In one embodiment, the spacing between adjacent filter pillars 112 of each filter array 104 ranges from about 0.5 μm to about 15 μm.

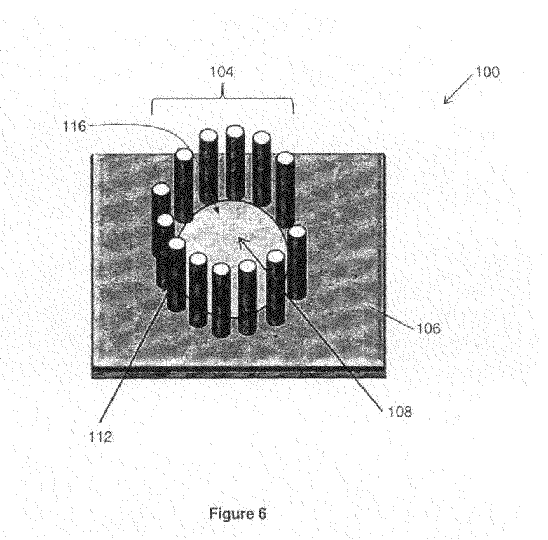

Further, as shown in FIG. 6, the filter pillars 112 of each filter array 104 may substantially surround a corresponding sensing electrode 108. In other words, each sensing electrode 108 may be arranged within the filter pillars 112 of the corresponding filter array 104. Each sensing electrode 108 may be disposed in the receptacle 116 formed by the filter pillars 112 of the corresponding filter array 104.

In one embodiment, each of the plurality of filter arrays 104 includes an insulating material. Examples of the insulating material can include silicon dioxide, silicon nitride and SU8. Each of the plurality of filter arrays 104 may include a conducting material. The conducting material may include but is not limited to nickel, gold and copper.

As shown in FIG. 1, the at least one support substrate 106 includes a plurality of through vias 118 arranged spaced apart from each other. The sensor array 102 further includes a plurality of interconnect portions 120. Each of the plurality of interconnect portions 120 is arranged within each of the plurality of through vias 118. Each of the plurality of interconnect portions 120 is electrically coupled to each of the plurality of sensing electrodes 108. In one embodiment, each of the plurality of interconnect portions 120 includes a conductive material. The conductive material may include but is not limited to gold, platinum and conducting polymer. The material used for the interconnect portions 120 and the material used for the sensing electrodes 108 may be similar. However, it is desirable to keep the interconnect portions 120 isolated as the sensing electrodes 108 are usually inert and biocompatible metals.

The microfludic device 100 further includes a cover layer 122 positioned over the plurality of filter arrays 104. The cover layer 122 may be formed integrally with the plurality of filter arrays 104. The cover layer 122 includes a plurality of openings 124. Each of the plurality of openings 124 is arranged to align with each of the plurality of sensing electrodes 108. The position of each opening 124 of the cover layer 122 is aligned with the position of the corresponding sensing electrode 108 within the filter array 104. Each of the plurality of openings 124 includes a dimension relative to the size of the single cell 114 being trapped within each of the plurality of filter arrays 104. In other words, each opening 124 has a dimension which allows the single cell to enter the receptacle 116 formed by the filter pillars 112 of the corresponding filter array 104.

The microfluidic device 100 may include antibody 128 placed within each of the plurality of filter arrays 104. The antibody 128 may be used to keep the cell 104 within each filter array 104 (i.e. in the receptacle 116 formed by the filter pillars 112 of each filter array 104).

In one embodiment, each of the plurality of sensing electrodes 108 is controlled by an electronic circuitry 126 built in the support substrate 106. The plurality of sensing electrodes 108 is electrically coupled to the electronic circuitry 126 via the corresponding interconnect portions 120.

The support substrate 106 may include silicon, gallium arsenide, gallium nitride and other semiconducting materials if the support substrate 106 includes built-in electronic circuits.

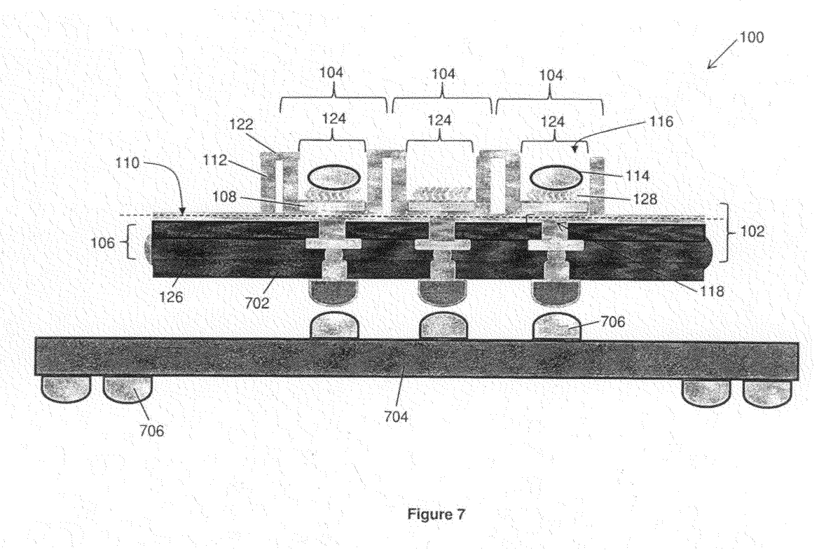

In another embodiment, as shown in FIG. 7, the microfluidic device 100 may include an integrated circuit 702 which is electrically coupled to the electronic circuitry 126. The microfluidic device 100 may be coupled to other devices via an external electrical interface 704 having a plurality of solder, balls 706.

The microfluidic device 100 has a microfabricated filter structure integrated with an electrode array. The microfluidic device 100 can be used for filtering of CTCs and electrical detection of CTCs. The microfluidic device 100 filters the CTCs using the filter arrays 104 by exploiting differences in size of CTCs from other cells found in blood, and enumerate the CTCs on the same structure. The cells are counted in the respective receptacles 116 which are equipped with corresponding sensing electrodes 108. The microfluidic device 100 can be used for label free enrichment and label free counting of CTCs. Label free cell enrichment of the microfluidic device 100 can enhance efficiency. The microfluidic device 100 can provide precise cell counting. Since the enrichment and counting of the cells are carried out in a single device without sample transfer, cell loss can be reduced.

The microfluidic device 100 is configured such that the sample flows in a vertical in, lateral out direction. This can allow integration of a large number of CMOS addressable electrodes in the flow path of the sample, thus allowing filtering, trapping (positioning) and electrical characterization of a wide range of numbers of cells (e.g. 0 to 10,000) on the same microfluidic device 100.

The microfluidic device 100 can preclude the use of optical imaging by integrating a high density electrode array with the filter for counting. Further, the microfluidic device 100 includes a high density electrode array integrated with the filter for counting. Thus, the microfluidic device 100 can independently address individual cells which are necessary for counting. The microfluidic device 100 can use through silicon via (TSV) array integrated with filter array for filtering CTCs based on their size and counting a large number of CTCs in a single step filtration and enumeration process.

The microfluidic device 100 can be used as an electrical/electromechanical sensor array for detection of cells from body fluids and/or tissue samples for diagnosis and monitoring purpose. The microfluidic device 100 can also be used for detection of CD4⁺ T lymphocytes for HIV, endothelial progenitor cells (EPCs) for cardiovascular related disease, CTCs for cancer, maternal fetal cell based Dx and microbial fuel cell.

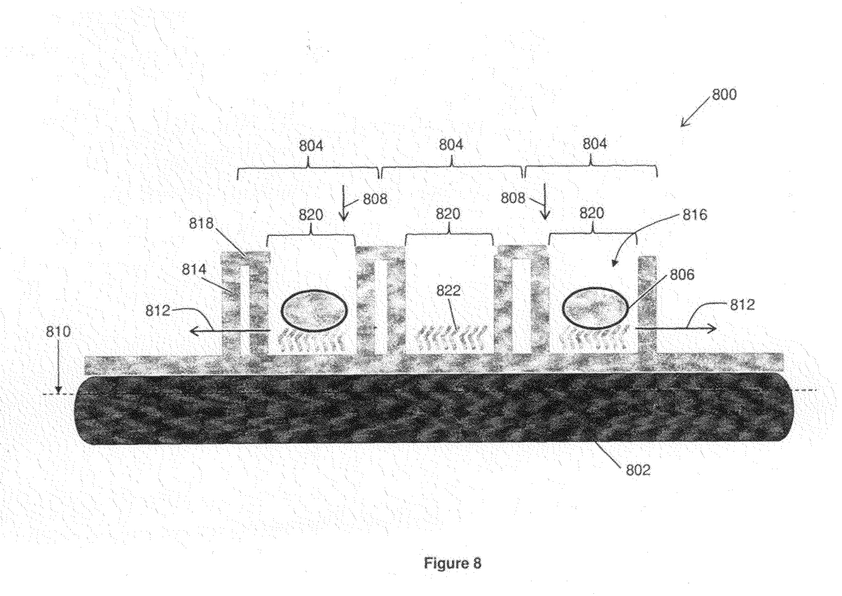

FIG. 8 shows a schematic diagram of a microfluidic device 800 according to one embodiment. The microfluidic device 800 may be used for single cell filtering. The microfluidic device 800 includes a support substrate 802 and at least one filter array 804 arranged on the support substrate 802. For illustration purposes, only three filter arrays 804 are shown. The filter arrays 804 and the support substrate 802 may be integrated.

Each filter array 804 is configured to trap a single cell 806 in contact with the support substrate 802 within the at least one filter array 804 when a sample (not shown) including a plurality of cells flows into the microfluidic device 800 from a direction 808 substantially perpendicular to a plane 810 of the support substrate 802 and flows out in a direction 812 substantially parallel to the plane 810 of the support substrate 802.

In one embodiment, each filter array 804 includes a plurality of spaced apart filter pillars 814 extending from the plane 810 of the support substrate 802. The plurality of spaced apart filter pillars 814 is configured to substantially surround the single cell 806 in contact with the support substrate 802. The filter pillars 814 of each filter array 804 may be arranged to form a receptacle 816 for receiving the single cell 806 respectively.

The spacing between the filter pillars 814 of each filter array 804 can allow passage of deformable erythrocytes, leukocytes and other smaller cells while retaining the CTCs in the receptable 806 due to their large size. In one embodiment, the spacing between adjacent filter pillars 814 of each filter array 804 ranges from about 0.5 μm to about 15 μm.

In one embodiment, the at least one support substrate 802 includes silicon, glass, polymer (e.g. PMMA, PDMS, polycarbonate) and other materials that can be used for microfabrication, microelectronics and microfluidics. The support substrate 802 may include silicon, gallium arsenide, gallium nitride and other semiconducting materials if the support substrate 802 includes built-in electronic circuits.

In one embodiment, each of the plurality of filter arrays 804 includes an insulating material. Examples of the insulating material can include silicon dioxide, silicon nitride and SU8. Each of the plurality of filter arrays 804 may include a conducting material. The conducting material may include but is not limited to nickel, gold and copper.

The microfluidic device 800 may further include a cover layer 818 positioned over the plurality of filter arrays 804. The cover layer 818 may be formed integrally with the plurality of filter arrays 804. The cover layer 818 includes a plurality of openings 820. Each of the plurality of openings 820 is arranged to align with each of the plurality of filter arrays 804. The position of each opening 820 of the cover layer 818 is aligned with the position of the corresponding receptacle 816 of the filter array 104. Each of the plurality of openings 820 includes a dimension relative to the size of the single cell 806 being trapped within each of the plurality of filter arrays 804. In other words, each opening 820 has a dimension which allows the single cell 806 to enter the receptacle 816 formed by the filter pillars 814 of the corresponding filter array 804.

The microfluidic device 800 may include antibody 822 placed within each of the plurality of filter arrays 804. The antibody 822 may be used to keep the cell 806 within each filter array 804 (i.e. in the receptacle 816 formed by the filter pillars 814 of each filter array 804).

Sensing of the cells 806 trapped by the plurality of filter arrays 804 can be performed by optical inspection from the top or bottom of the microfluidic device 800.

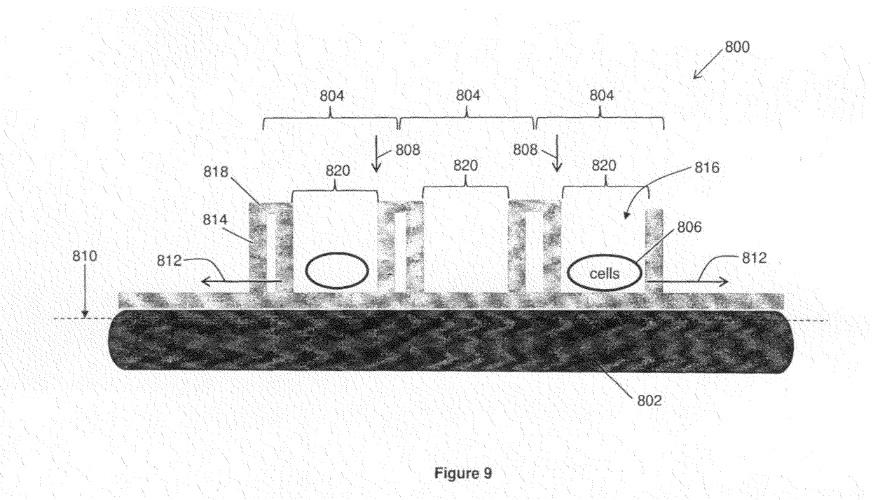

In one embodiment, as shown in FIG. 9, the plurality of filter arrays 804 can be used to keep the single cell 806 within each of the plurality of filter arrays 804 (i.e. the receptacle 816 formed by the filter pillars 814 of each filter array 804). As such, antibodies are not used.

It may be possible to trap more than one cell 806 within each filter array 804 by adjusting the dimensions of the filter array 804 (i.e. the receptacle 816 formed by the filter pillars 814 of each filter array 804) according to the size of the cell 806.

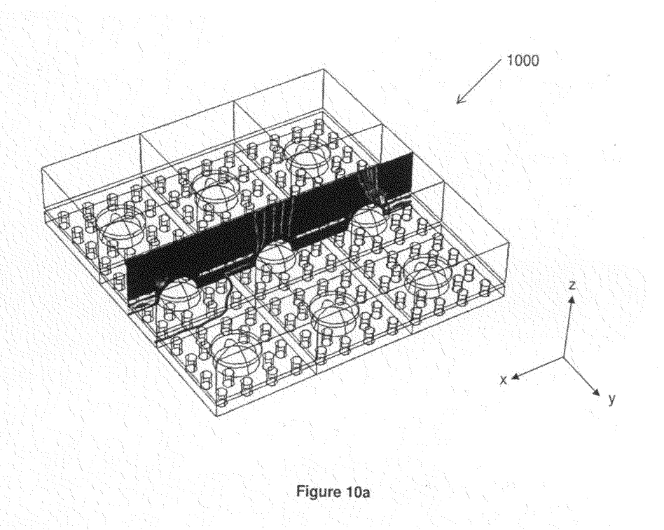

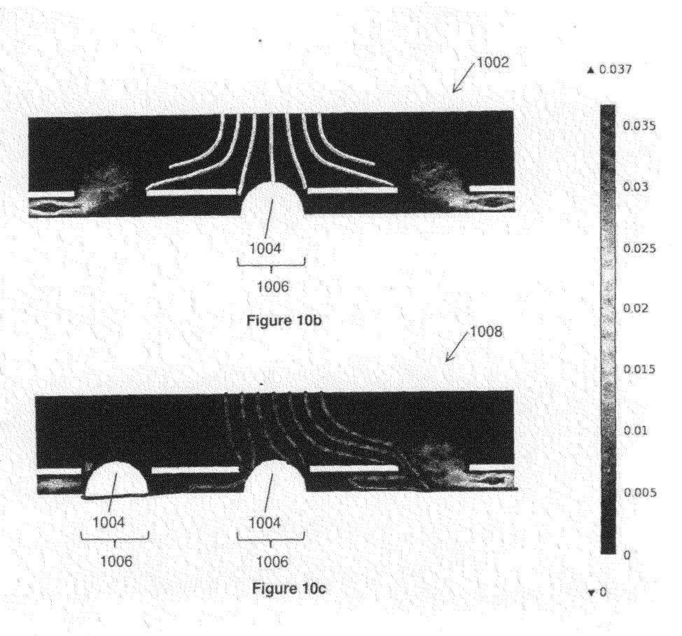

FIG. 10a shows a three-dimensional view of a microfluidic device 1000 for simulation. FIG. 10b shows a simulated flow velocity profile 1002 of a sample in the microfluidic device 1000 along the x-z plane when a cell 1004 is trapped within one filter array 1006. FIG. 10c shows a simulated flow velocity profile 1008 of a sample in the microfluidic device 1000 along the x-z plane when a cell 1004 is trapped within two filter arrays respectively.

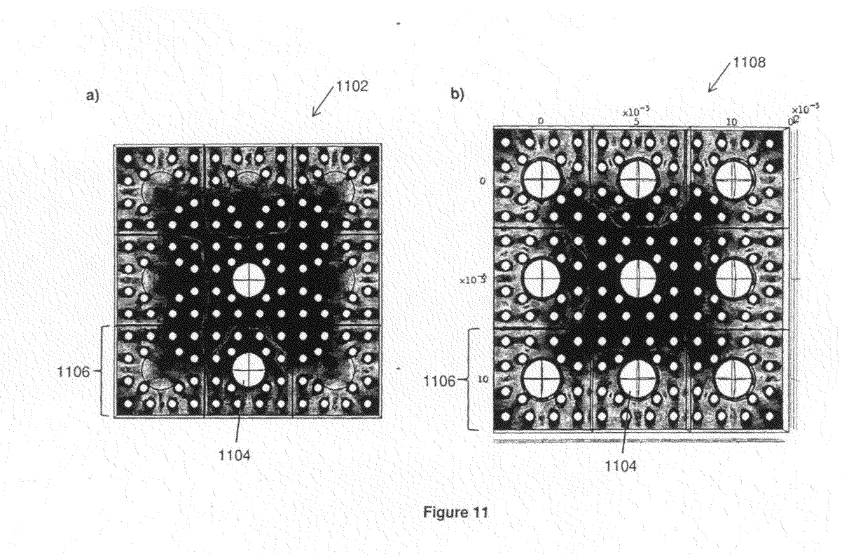

FIG. 11a shows a simulated flow velocity profile 1102 of a sample in the microfluidic device 1000 along the x-y plane when a cell 1104 is trapped within two filter arrays 1106 respectively. FIG. 11b shows a simulated flow velocity profile 1108 of a sample in the microfluidic device 1000 along the x-y plane when a cell 1104 is trapped within all filter arrays 1106 respectively. A maximum flow velocity can be observed at the periphery of the microfluidic device 1000.

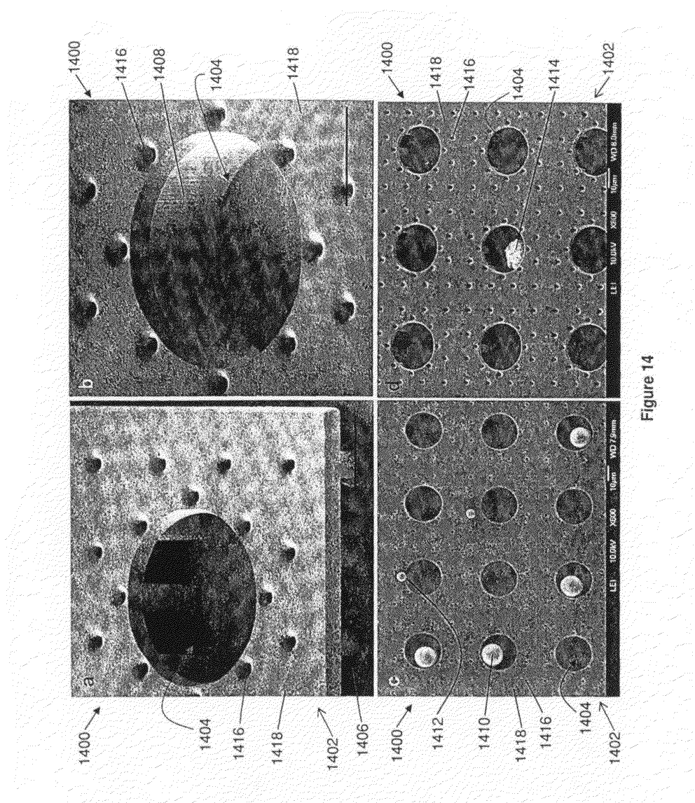

FIGS. 14a to 14d show scanning electron micrographs (SEM) of a microfluidic device 1400 according to one embodiment (scale bar is 10 μm). FIG. 14a shows a fabricated filter array 1402 of the microfluidic device 1400. The filter array 1402 may include a 24 μm diameter micro-well (e.g. receptacle) 1404 supported by filter pillars 1406. FIG. 14b shows a micro-well structure (e.g. receptacle) 1404 prior to removal of a filler material 1408. FIG. 14c shows 15 μm beads 1410 and 8 μm beads 1412 trapped within the filter array 1402. FIG. 14d shows a single Jurkat cell 1414 isolated in the filter array 1404. Dimples 1416 on a capping membrane (e.g. cover layer) 1418 shown in FIGS. 14a to 14d may be resulted from a fabrication process of the microfluidic device 1400.

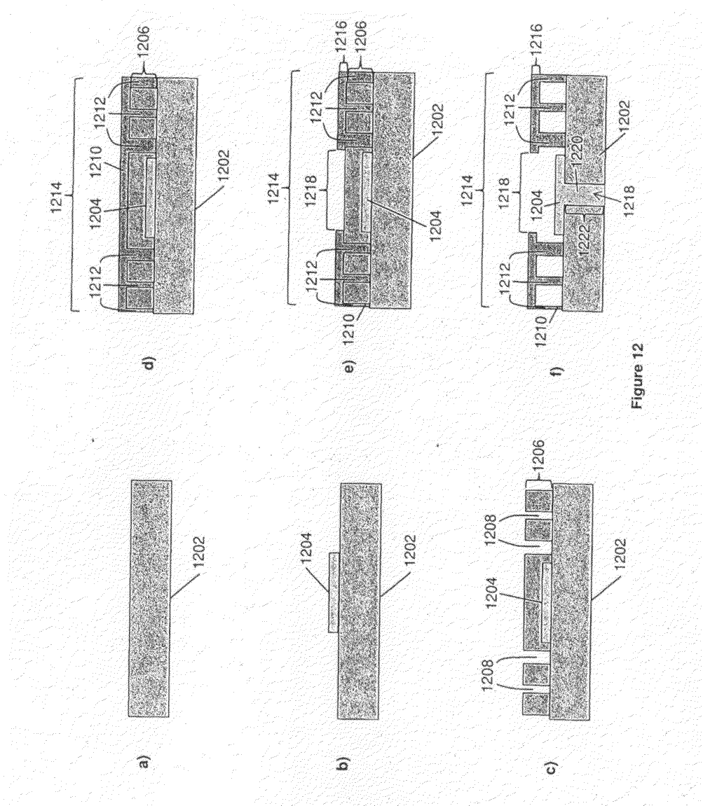

FIGS. 12a to 12f show an exemplary process of manufacturing a microfluidic device. FIG. 12a shows a support substrate 1202. The support substrate 1202 may include silicon, glass, polymer (e.g. PMMA, PDMS, polycarbonate) and other materials that can be used for microfabrication, microelectronics and microfluidics if the support substrate 1202 does not include any built-in electronic circuits. The support substrate 1202 may include silicon, gallium arsenide, gallium nitride and other semiconducting materials if the support substrate 1202 includes built-in electronic circuits.

FIG. 12b shows that a sensing electrode 1204 is formed on the support substrate 1202. The sensing electrode 1204 may include conducting materials such as gold, platinum and conducting polymer or semiconducting materials such as silicon.

FIG. 12c shows that a sacrificial layer 1206 is deposited above the support substrate 1202 and the sensing electrode 1204. The sacrificial layer 1206 is etched to form cavities 1208. The sacrificial layer 1206 may include silicon dioxide, parylene, photoresist and other polymer materials that can be easily removed by wet or dry etching.

FIG. 12d shows that an insulating material 1210 is deposited above the sacrificial layer 1206 and is deposited in the cavities 1208 to form pillar structures 1212 of a filter array 1214. The insulating material 1210 may include silicon dioxide, silicon nitride and SU8. Alternatively, a conducting material such as nickel, gold and copper can be used for forming the pillar structures 1212.

FIG. 12e shows that the insulating material 1210 is etched to form a cover layer 1216 with an opening 1218. The opening 1218 of the cover layer 1216 is arranged to align with the sensing electrode 1204. The opening 1218 may have a dimension relative to the size of a single cell being trapped within the filter array 1214. In other words, the opening 1218 has a dimension which allows the single cell to enter the filter array 1214.

FIG. 12f shows that the sacrificial layer 1206 is removed and the support substrate 1202 is etched to form a through via 1218. A conducting material 1220 is deposited in the through via 1218 to form an interconnect portion 1222. The conducting material 1220 may include gold, platinum and conducting polymer or semiconducting materials such as silicon. The interconnect portion 1222 allows the microfluidic device to be electrically coupled to a chip/integrated circuit.

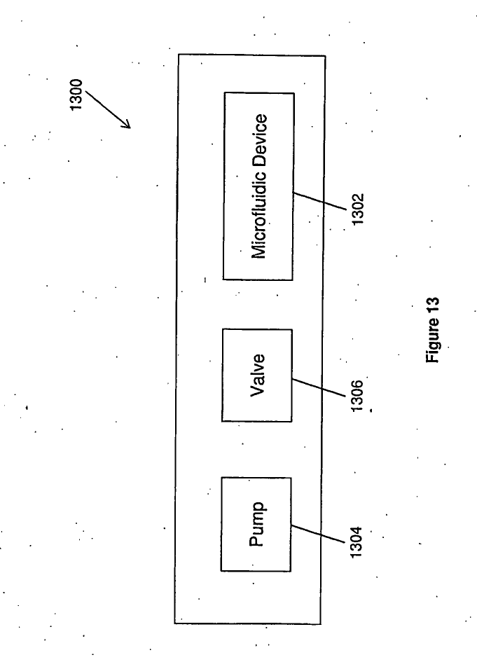

FIG. 13 shows a schematic diagram of a microfluidic system 1300. The microfluidic system 1300 may include a microfluidic device 1302, at least one pump 1304 and at least one valve 1306. For illustration purposes, one pump 1304 and one valve 1306 are shown in FIG. 13. The microfluidic device 1302 may correspond to the microfluidic device 100. The at least one pump 1304 and at least one valve 1306 are configured to allow, a sample including a plurality of cells to be pumped into the microfluidic device 1302.

While embodiments of the invention have been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

1. A microfluidic device for single cell filtering, the microfluidic device comprising:
a support substrate; and
at least one filter array arranged on the support substrate and configured so as to trap a single cell in contact with the support substrate within the at least one filter array when a sample including a plurality of cells flows into the microfluidic device from a direction substantially perpendicular to a plane of the support substrate and flows out in a direction substantially parallel to the plane of the support substrate.

2. The microfluidic device of claim 1, wherein the at least one filter array comprises a plurality of spaced apart filter pillars extending from the plane of the support substrate and configured to substantially surround the single cell in contact with the support substrate.

3. A microfluidic device for single cell filtering and detection, the microfluidic device comprising:
a sensor array comprising at least one support substrate and a plurality of sensing electrodes arranged spaced apart on the at least one support substrate; and
a plurality of filter arrays arranged on the sensor array, each of the plurality of filter arrays arranged corresponding to each of the plurality of sensing electrodes and configured to trap a single cell in contact with each of the plurality of sensing electrodes within each of the plurality of filter arrays when a sample including a plurality of cells flows into the microfluidic device in a direction substantially perpendicular to a plane of the at least one support substrate and flows out in a direction substantially parallel to the plane of the at least one support substrate.

4. The microfluidic device of claim 3, wherein the at least one support substrate and the plurality of filter arrays are integrated.

5. The microfluidic device of claim 3, wherein the at least one support substrate comprises a plurality of through vias arranged spaced apart from each other.

6. The microfluidic device of claim 5, wherein the sensor array further comprises a plurality of interconnect portions, each of the plurality of interconnect portions is arranged within each of the plurality of through vias.

7. The microfluidic device of claim 6, wherein each of the plurality of sensing electrodes is electrically coupled to each of the plurality of interconnect portions.

8. The microfluidic device of claim 6, wherein each of the plurality of sensing electrodes is controlled by an electronic circuitry built in the support substrate.

9. The microfluidic device of claim 3, wherein each of the plurality of filter arrays comprises a plurality of spaced apart filter pillars extending from the plane of the at least one support substrate and configured to substantially surround the single cell in contact with each of the plurality of the sensing electrodes.

10. The microfluidic device of claim 9, further comprising a cover layer positioned over the plurality of filter arrays, the cover layer comprising a plurality of openings, each of the plurality of openings arranged to align with each of the plurality of sensing electrodes.

11. The microfluidic device of claim 10, wherein each of the plurality of openings includes a dimension relative to the size of the single cell being trapped within each of the plurality of filter arrays.

12. The microfluidic device of claim 3, wherein the at least one support substrate comprises a plurality of support substrates arranged in a stack.

13. The microfluidic device of claim 12, further comprising an adhesive layer positioned between adjacent support substrates of the plurality of support substrates.

14. The microfluidic device of claim 13, wherein the adjacent substrates of the plurality of support substrates are bonded together by means of the adhesive layer.

15. The microfluidic device of claim 3, wherein the at least one support substrate comprises silicon, glass, or polymer.

16. The microfluidic device of claim 3, wherein each of the plurality of interconnection portions comprises a conductive material.

17. The microfluidic device of claim 3, wherein each of the plurality of filter arrays comprises an insulating material.

18. A microfluidic system comprising:
the microfluidic device of claim 3;
at least one pump; and
at least one valve;
wherein the at least one pump and the at least one valve is configured to allow a sample including a plurality of cells to be pumped into the microfluidic device.
